(19) United States
(12) Patent Application Publication
(10) Pub. No.: US 2014/0276814 A1
(43) Pub. Date: Sep. 18, 2014

(54) TISSUE RESECTION DEVICE AND RELATED METHODS OF USE

(71) Applicant: BOSTON SCIENTIFIC SCIMED, INC., Maple Grove, MN (US)

(72) Inventors: Paul SMITH, Smithfield, RI (US); Samuel RAYBIN, Marlborough, MA (US); Naroun SUON, Lawrence, MA (US); Michael LYNN, Braintree, MA (US)

(73) Assignee: BOSTON SCIENTIFIC SCIMED, INC., Maple Grove, MN (US)

(21) Appl. No.: 14/204,615

(22) Filed: Mar. 11, 2014

Related U.S. Application Data
(60) Provisional application No. 61/784,876, filed on Mar. 14, 2013.

(51) Int. Cl. A61B 17/3205; A61B 18/14

(52) U.S. Cl. CPC A61B 17/32056; A61B 18/1482
USPC 606/49; 606/110; 606/113

(57) ABSTRACT

A medical device may include tubular member having a proximal end, a distal end, and a lumen extending therebetween. The tubular member may include an opening at a distal portion of the tubular member, wherein the opening is in communication with the lumen, wherein a distal end of the opening is disposed proximate of the distal end of the tubular member, and wherein the distal portion of the tubular member is configured to bend in at least one plane. The medical device may further include an actuation member extending at least partially within the tubular member, wherein a distal portion of the actuation member is configured to exit the tubular member at a location proximate the opening, wherein a distal end of the actuation member is coupled to the distal end of the tubular member, and wherein pulling the actuation member proximally is configured to cause the distal portion of tubular member to form a loop with itself.

### CROSS-REFERENCE TO RELATED APPLICATIONS

This application claims the benefit of priority from U.S. Provisional Application No. 61/784,876, filed on Mar. 14, 2013, the entirety of which is incorporated by reference herein.

### FIELD OF THE DISCLOSURE

The present disclosure relates generally to medical devices and procedures. More particularly, exemplary embodiments of the disclosure relate to devices and methods for removing unwanted tissue (e.g., lesions) by, e.g., resection or dissection.

### BACKGROUND

A wide variety of medical techniques and instruments have been developed for diagnosis and/or treatment within a patient's body, such as within a patient's Gastrointestinal (GI) tract. Endoscopic Mucosal Resection (EMR), Endoscopic Sub-mucosal Resection (ESR), Polypectomy, Mucosectomy, etc., are minimally invasive treatment methods for both malignant and non-malignant lesions. Endoscopic medical procedures, such as, for example, EMR, may be used to excise sessile adenomas or other unwanted tissue (i.e., tumors attached to a bodily surface) from the surface of an anatomical lumen. Such procedures often require the resection of one tissue plane while leaving an underlying tissue plane intact. Commonly, snares, designed as loops, have been used during such medical procedures, for resecting tissue from a target site. However, the conventional snares have a tendency to slip off the targeted tissue, and often require repeated efforts to capture the tissue before the resection procedure(s) can be successfully performed.

To address the slippage problem, one way is to apply a downward force on the snare in an effort to improve traction between the snare and the unwanted or target tissue. This downward force is usually limited due to a lack of stiffness in the snare loop, however, causing the distal end of the snare loop to deflect away from the targeted tissue, thereby leading to improper placement of the snare. To control the deflection, the downward force may continue to be applied or it may be increased until the tissue is snared. Continued or increased applied force increases the possibility of accidentally damaging surrounding tissues (in particular underlying tissue layers, such as, e.g., the muscularis), as well as increasing the time and effort required to complete a procedure. Furthermore, it may sometimes be difficult to apply the necessary downward forces due to geometric and physical limitations.

Many standard cutting tools are used at perform tissue resection. However, at times, application of these standard cutting tools may result in inadvertent damage to surrounding tissue and/or underlying tissue layers. This is because the tools are typically incapable of adequately controlling the depth of penetration into the target site, during operation, and further, are sometimes incapable of adequately distinguishing between different types of tissues or tissue layers. These characteristics can be highly undesirable, and may cause loss of blood and/or healthy tissue from a patient's body, thus, again resulting in visualization difficulties for a physician.

Therefore, there exists a need for a device having better control capabilities, and which can effectively encapsulate and then resect tissue without damaging surrounding tissues and/or underlying tissue layers of an organ or other target tissue while effectively and efficiently excising unwanted tissue.

### SUMMARY OF THE DISCLOSURE

The present disclosure relates to embodiments of medical devices and relates methods for resecting unwanted tissue from a portion of a human body.

An embodiment of the present disclosure includes a medical device comprising a tubular member having a proximal end, a distal end, and a lumen extending therebetween, the tubular member including an opening at a distal portion of the tubular member, wherein the opening is in communication with the lumen, wherein a distal end of the opening is disposed proximate of the distal end of the tubular member, and wherein the distal portion of the tubular member is configured to bend in at least one plane. The medical device may further include an actuation member extending at least partially within the tubular member, wherein a distal portion of the actuation member is configured to exit the tubular member at a location proximate the opening, wherein a distal end of the actuation member is coupled to the distal end of the tubular member.

In various embodiments, the medical device may also include: a cutting tool disposed within the opening; wherein the opening includes an elongate configuration; wherein the cutting tool is an electro-cautery tool; wherein the cutting tool is configured to traverse the opening; wherein the lumen is configured to be coupled to a suction source; wherein the opening extends distally from the location where the actuation member exits the tubular member; wherein an external surface of the distal portion includes a plurality of radiopaque markings; wherein the cutting tool is slidably disposed on a track that traverses the opening; wherein pulling the actuation member proximally is configured to cause the distal portion of tubular member to form a loop with itself.

Another embodiment of the present disclosure includes a medical device including an elongate tubular member having a proximal end, a distal end, and a lumen extending therebetween, wherein a distal end portion of the elongate tubular member is configured to bend towards a remaining portion of the elongate member to create a loop-like configuration, and wherein the distal end portion includes an elongate rectangular opening in communication with the lumen. The medical device also may include an actuation member coupled to a distal end face of the tubular member, wherein pulling the actuation member proximally causes the distal end portion of the elongate tubular member to bend towards the remaining portion.

In various embodiments, the medical device may also include: a cutting tool disposed within the opening; wherein the cutting tool is an electro-cautery tool; wherein the cutting tool is configured to traverse the opening; wherein the lumen is configured to be coupled to a suction source; wherein a portion of the actuation member is disposed within the elongate tubular member, and a portion of the actuation member is disposed outside of the elongate tubular member; wherein an external surface of the distal portion includes a plurality of radiopaque markings; and wherein the cutting tool is slidably disposed on a track that traverses the opening.

In an exemplary embodiment, a method of resecting tissue may include positioning a medical device adjacent tissue targeted for resecting. The medical device may include an elongate tubular member having a proximal end, a distal end, and a lumen extending therebetween, wherein a distal end portion of the elongate tubular member is configured to bend towards a remaining portion of the elongate member to create a loop-like configuration, and wherein the distal end portion includes an elongate rectangular opening in communication with the lumen. The medical device also may include an actuation member coupled to a distal end face of the tubular member, wherein pulling the actuation member proximally causes the distal end portion of the elongate tubular member to bend towards the remaining portion of the elongate tubular member. The method may further include pulling the actuation member to bend the distal end portion towards the elongate tubular member to create a substantially loop-like configuration; drawing tissue into the elongate rectangular opening, and activating a cutting tool to cut through the tissue drawn into the elongate rectangular opening. The tissue drawn into the elongate rectangular opening may include only a mucosal layer of tissue. The method may also include retrieving the resected tissue from within the patient's body.

Additional characteristics and advantages of the described embodiments will be set forth in part in the description that follows, and in part will be apparent from the description, or, may be learned by practicing the disclosure. The characteristics and/or advantages of the disclosure may be realized and attained by way of the elements and combinations particularly pointed out in the appended claims.

It is to be understood that both the foregoing general description and the following detailed description are exemplary and explanatory only, and are not restrictive of the described embodiments or the claimed subject matter.

### BRIEF DESCRIPTION OF THE DRAWINGS

The accompanying drawings, which are incorporated in and constitute a part of this specification, illustrate exemplary embodiments of the present disclosure and, together with the description, serve to explain the principles of the disclosure.

FIG. 1A illustrates an exemplary medical device employing a tubular member, according to an embodiment of the present disclosure.

FIG. 1B is a schematic view of the tubular member shown in FIG. 1A.

FIG. 2 is a schematic illustration of the tubular member of FIG. 1A in a closed configuration, in accordance with the present disclosure.

FIG. 3A shows a cutting tool positioned inside a suction channel of the tubular member, according to the present disclosure.

FIG. 3B illustrates an embodiment of operating the cutting tool of FIG. 3A, in accordance with the present disclosure.

FIG. 3C is an alternative embodiment of a cutting tool, in accordance with a further embodiment of the present disclosure.

FIG. 4A is a schematic view of an exemplary device placed around a target tissue layer, according to the present disclosure.

FIG. 4B is a schematic view of a curved incision made on the tissue layer to be resected, in accordance with the present disclosure.

FIG. 4C shows the use of a snare device for resecting and/or retrieving the resected tissue layer from within a patient's body, in accordance with the present disclosure.

### DETAILED DESCRIPTION OF EXEMPLARY EMBODIMENTS

Reference will now be made in detail to exemplary embodiments of the present disclosure, examples of which are illustrated in the accompanying drawings. Wherever possible, the same reference numbers are used throughout the drawings to refer to the same or like parts. The term “distal” refers to the end farthest away from a user when introducing a device into a patient. The term “proximal” refers to the end closest to the user when placing the device into the patient.

### Overview

Embodiments of the present disclosure relate to devices and methods for resecting or otherwise cutting or excising and retrieving/removing undesirable or target tissues from a patient's body. For example, the device may remove cancerous polyps or lesions from the mucosal walls of the colon, esophagus, stomach, duodenum, or any other suitable location. It should be understood that the resection device may perform the functions of both resecting and retrieving, but for convenience, the term “resection device” will be used throughout this application.

In the following sections, embodiments of the present disclosure will be described using an exemplary body organ—the stomach. The embodiments of the resection device discussed below aim to resect and/or remove tissue from the mucosal layer of the stomach without damaging the underlying tissue layers, including, but not limited to, the muscularis. It will be understood that the stomach is merely exemplary and that the device may be utilized in any other suitable organ, and in particular the gastrointestinal (GI) tract, such as the colon, duodenum, esophagus, or any other organ that may be subject to polyps, lesions, or that requires tissue resection or biopsy.

The present disclosure is directed to a tissue resection device and method that overcomes or addresses, among other things, the shortcomings mentioned above and other shortcomings known to those in the art. The disclosed tissue resection device separates a tissue layer intended for resection from sub-tissue layers and surrounding tissue not intended for resection. For example, in one embodiment, the device may be configured to separate the mucosa and muscularis layers so that resecting a lesion on the mucosal layer does not perforate the muscularis layer. More particularly, the device creates an approximately circular incision at a safe depth to facilitate resection of the encircled tissue. The incision thus formed can facilitate the capture of the tissue layer without slippage, using various tools. The disclosed device is designed to be used with an apparatus like an endoscope or any other suitable introduction sheath known in the art.

In view of the above, a device for tissue resection and/or retrieval is disclosed, which includes, among other things, a tubular member, an actuation member and a cutting tool. The tubular member may have a proximal end, a distal end, and a lumen extending therebetween. A slot or other opening located at the distal end, may extend longitudinally along a length of the tubular member, thereby forming a channel including a proximal end and a distal end. The channel may be a suction channel. The actuation member is coupled to the distal end, and may be actuated proximally such that the distal end of the tubular member moves (e.g., bends) towards the proximal end of the suction channel, creating a distal loop, to encircle a tissue layer. Additionally, the cutting tool, which may be disposed inside the channel, may be activated (e.g., advanced along the channel) to cut the tissue encircled by the tubular member, as described in greater detail below. Herein, the cutting tool may be configured only to make an incision in a desired tissue layer (e.g., a mucosal layer) while leaving an underlying layer (e.g., a muscularis layer) intact, or may be configured to make both the incision and resection.

### Exemplary Embodiments

FIG. 1A shows an exemplary device 100, employing a tubular member 102. The device 100 may be configured to be introduced into a patient's body through a suitable natural opening or an incision. Specifically, the device 100 may be configured to be advanced to a desired location within a patient's body with the aid of a suitable introduction sheath, such as, e.g., an endoscope 10 that can further include a steerable elongate sheath 12 having a distal end 14, a proximal end (not shown) and one or more working channels extending therebetween.

As discussed above, the endoscope 10 may include one or more working channels, wherethrough an operator may introduce one or more medical devices to extend out of the distal end 14 of elongate sheath 12. For example, during a resection procedure, an operator may introduce a device such as resection device 100 in one channel, and any assistive surgical instrument into another channel. The other working channels may be used for facilitating visualization, illumination, irrigation and/or insufflation of the surgical site. In addition, one of the other working channels may be used to provide suction or other suitable means (e.g., retrieval tools) for capturing and retrieving excised tissues from within the patient. The proximal end of the elongate sheath 12 may be connected to a handle (not shown) for operating the endoscope 10. The handle may be ergonomically designed and may include a variety of components such as steering controls for selectively positioning the distal end 14. Also, the handle may include one or more ports in communication with the one or more working channels for inserting the one or more medical devices into the endoscope 10.

Resection device 100 may be typically carried within a working channel of the endoscope 10, during the time when the elongate sheath 12 is being navigated through a patient's body. In another embodiment, elongate sheath 12 may be first positioned at a targeted site, and then device 100 may be inserted through a working channel to protrude from a distal end of elongate sheath 12. Insertion within the working channel requires flexibility in certain portions of the resection device 100. Moreover, the resection device 100 may be defined as a device used for resecting and/or retrieving an unwanted tissue layer or other material inside the body of a patient. As shown in FIG. 1A, the resection device 100 may include a tubular member 102, an actuation member 104, and a cutting tool 302 (see FIG. 3A). The working channel and tubular member 102 may be shaped and sized for placement into a patient via a body cavity or an incision. The diameter of the tubular member 102 may be smaller than the diameter of the working channel. In some embodiments, the diameter of the working channel may be about 3.5 to 4.0 centimeters and the diameter of the tubular member 102 is about 3.0 to 3.4 centimeters. As used herein, about means plus or minus 10 to 20 percent the stated dimensions. In one embodiment, the diameter of the tubular member may be 3.5 centimeters. Each component will now be discussed in detail in the following sections.

The tubular member 102 may include a proximal end (not shown), a distal end 106, and an outer surface. The tubular member may further include a lumen 108 defined between the proximal end and the distal end 106. The lumen 108 may extend through the entire length of the tubular member 102 and may be configured to slidably receive one or more medical devices such as the cutting tool 302, a snare, a basket, a forcep, a grasper, etc. In some embodiments, lumen 108 may extend through a only a portion of the length of tubular member 102. For example, lumen 108 may only extend within a distal portion of tubular member 102. Further, a proximal end of lumen 108 may be either closed or open as desired. In some embodiments, the tubular member 102 may include a number of lumens (instead of only one lumen 108) and/or corresponding openings for a variety of purposes, such as inserting additional medical devices, deploying a snare device, or the like. The tubular member 102 may be configured to be steerable independently of the endoscope 10 using any actuation mechanism. As distal portion of tubular member 102 may be formed of a plurality of interconnected articulating joints, which may facilitate bending or steering the distal portion of tubular member 102, as described in greater below. A plurality of pull-wires may be connected to the plurality of interconnected articulating joints for moving the joints relative to another.

The distal end portion of the tubular member 102 may be flexible enough to be deflected/bent relative to a longitudinal axis of the tubular member 102. Such deflection may be produced by the actuation member 104, for example. Once the tubular member 102 takes the desired shape, the rigidity of the member 102 ensures that the shape is maintained during the operation. In addition, proximal end of the tubular member 102 may be connected to a handle (not shown) and this will be discussed below in detail. In some embodiments, the bendable distal end portion of tubular member 102 may include radiopaque markers or be otherwise configured to facilitate visualization within a patient's body.

During operation, the tubular member 102 may be configured to move between a straightened configuration, where the tubular member 102 may be advanced through elongate sheath 12 for placement adjacent tissue targeted for resection, and a closed configuration, in which the tubular member 102 may be configured to encircle the target tissue layer, for resection. As shown in, e.g., FIG. 2, in the straightened configuration, an entirety of tubular member 102 may be disposed along its longitudinal axis. In the second, closed configuration, a distal portion of tubular member 102 may be bent or steered backwards so that tubular member 102 is curved upon itself.

The tubular member 102 may be flexible along its entire length or adapted for flexure along portions of its length. Further, certain portions of tubular member 102 may be more flexible than other portions. For example, a distal portion of tubular member 102 may include a flexibility greater than the flexibility of a proximal or central portion of tubular member 102. Alternatively, or in addition, the distal end 106 may be flexible while the remaining tubular member 102 may be rigid. Flexibility allows the member 102 to maneuver turns in body lumens and/or to be deflected into the desired position, while rigidity provides the necessary force to urge the member 102 forward. In the example shown, the tubular member 102 may be a long, flexible or a semi-flexible, structure having a circular cross-section. Alternatively, other suitable cross-sections such as, but not limited to, rectangular, triangular, oval, or irregular, or the like may also be contemplated. The cross-sectional configuration of the tubular member 102 may be uniform or may vary along its length.

Further, the tubular member 102 may include an opening such as, e.g., a slot 109, disposed at the distal end portion of tubular member 102. In some embodiments, slot 109 may include a substantially elongate rectangular configuration, as depicted in FIG. 1A. That is, slot 109 may extend longitudinally along a length of the tubular member 102. In other embodiments, slot 109 may include an oval or circular configuration. Those of ordinary skill in the art will recognize that the shape and configuration may be varied as desired. The slot 109 may extend through the outer surface of the tubular member 102 such that slot 109 may be in communication with the lumen 108 of the tubular member 102. The size and shape of the slot 109 may be based, at least in part, on the size and geometry of the tubular member 102, and may be varied as desired or to suit a particular application. In some embodiments, as shown in FIG. 2, in the closed configuration, the diameter of the loop formed by tubular member 102 may be about 1 to 2 centimeters. The length of the slot 109 may be calculated by the formula pi (π) multiplied by the diameter of the loop. In one embodiment, a lesion targeted for incision may have a diameter of about 1.5 centimeters. The length of slot 109 may be configured to encircle the targeted lesion. In an embodiment where the diameter of the loop formed by tubular member 102 may be about 1.5 centimeters, the length of slot 109 may be about 4.71 centimeters. Thus, in one embodiment, the ratio of the length of slot 109 to the diameter of the loop formed by tubular member 102 may be about 3. As used herein, about means plus or minus 10 to 20 percent the stated dimensions.

Specifically, the slot 109 may be cut at an angle to the longitudinal axis of the tubular member 102, creating a channel 111. The channel 111 may be used to deliver suction forces to the targeted tissue. The channel 111 may have a proximal end 113, distal end 115 and may be in communication with the lumen 108 of the tubular member 102. This will be further discussed in detail below.

As shown, the actuation member 104 (for example, a pull wire) may extend at least partially through the tubular member 102. More specifically, a portion of the actuation member 104 may extend externally from the distal end 106 of the tubular member 102, and may enter into the tubular member 102 through an opening 110 disposed adjacent the proximal end 113 of the channel 111. In some embodiments, the actuation member 104 may extend entirely along an outer surface of the tubular member 102. The actuation member 104 may be utilized to move or bend the distal end 106 of the tubular member 102 in a manner that causes a distal end portion of tubular member 102 to be curved towards a proximal portion of tubular member 102. Actuation member 104 may be configured to transmit both tensile and compressive forces to distal end 106 of tubular member 102.

More particularly, the actuation member 104 and the tubular member 102 may be arranged in a way such that actuating the actuation member 104 moves or bends the a distal end portion of the tubular member 102 so as disposed distal end 106 adjacent the proximal end 113 of the channel 111, thereby forming a loop 200 (see FIG. 2), which may be used to encircle tissue targeted for resection during operation of the tissue resection device 100.

As shown in FIG. 1A, the actuation member 104 may be a pull wire, but other known examples of the actuation member 104 are also contemplated. In some embodiments, actuation member 104 may include a rod or multiple pull wires, which may or may not be braided together.

Moreover, the actuation member 104 may have a proximal end connected to a handle (not shown) at the proximal end of the tubular member 102, and a distal end 105 detachably coupled to the distal end 106 of the tubular member 102 via any suitable means known in the art. In some embodiments, distal end 105 of actuator member 104 may be permanently coupled or integrally formed with distal end 106 of tubular member 106. The actuation member 104 may be controlled manually or automatically by an operator of the handle or similar mechanism at the proximal end of the tubular member 102.

As mentioned above, the channel 111 may be configured to apply suction to a tissue layer to pull the tissue layer into or adjacent the channel 111. During operation of the device, suction may be supplied via a suction source 116 connected to the proximal end of the tubular member 102. The suction source 116 may be configured to maintain an appropriate amount of low pressure within the lumen 108 of the tubular member 102, and the maintained pressure may be sufficient to separate one or more desired layers of tissue at the target site from other underlying layers. As an operator activates the suction source 116, the suction source 116 may initiate suction of air from the target site at a specific flow rate by creating a low pressure region within the lumen 108. This suction assists in pulling tissue at the target site into the suction 111, where a cutting tool, such as, e.g., cutting tool 302, may be positioned to accomplish cutting operation, according to the principles of the present disclosure. Those of ordinary skill in the art will understand that any suitable cutting or resection device may be used in accordance with the present disclosure. For example, in some embodiments, a discrete cutting tool may be advanced to the target tissue through a working channel of sheath 12 to cut tissue encircled by tubular member 102.

The channel 111 may be narrow such that the non-targeted sub tissue layer is excluded from being drawn up into the channel 111, thereby reducing the risk of perforation. A relatively narrow suction channel 111 may function to limit or control the amount of tissue drawn into the channel 111, thereby limiting the tissue drawn into channel 111 to only the upper tissue layer or layers at the targeted site. Additionally, the size of channel 111 may vary depending on the particular medical procedure and anatomy.

Additionally, the channel 111 may include a longitudinal track 114, extending from the proximal end 113 of the channel 111 to the distal end 115 of the channel 111. The track 114 may be permanently or temporarily fixed inside the channel 111. For example, the longitudinal track 114 may be adhesively connected, sealed or bonded to the channel 111. The longitudinal track 114 may be provided to hold a cutting tool, such that the cutting tool may move along and be guided by the track 114 inside the suction channel 111. The cutting tool and its operation will be further explained in conjunction with FIGS. 3A-3C. In some embodiments, track 114 may include one or more rails upon which a cutting device may slide or be guided through channel 111.

As discussed above, the handle (although not shown) at the proximal end of the tubular member may be configured to advance the tubular member 102 relative to the elongate sheath 12. Also, the handle may include the ability to steer the distal end 106 of the tubular member 102. The handle may further include one or more ports to introduce medical devices (such as suction source 116) into the working channel or the lumen 108 of the tubular member 102. Moreover, the handle may include an actuating mechanism to actuate one or more medical devices, such as the actuation member 104, the cutting tool 302, or others. In addition, the handle may include a mechanism to power on or off the suction source 116 attached at the proximal end.

Material employed to manufacture the tubular member 102 may include, but are not limited to rigid, flexible, or semi-rigid materials. Exemplary materials may include, polymers, composites, or the like. The chosen material may be based on desired stiffness, resilience, and other properties, as will be understood by those skilled in the art to be biocompatible. The tubular member 102 may be made of such material that provides a required stiffness to the loop while it is in the closed configuration and preferentially assumes a set shape when expanded, such as a circle or ellipse. The tubular member 102 can be made of the same or different types of materials, including those mentioned above, based on the degree of flexibility required for accessing the targeted tissue.

The tubular member 102 and its associated components may be coated with a suitable friction reducing material such as TEFLON®, polyetheretherketone, polyimide, nylon, polyethylene, or other lubricious polymer coatings, to reduce surface friction with the surrounding tissues. Alternatively, the tubular member 102 may be made of, or covered with, an insulating layer, to prevent inadvertent cauterizing of surrounding tissue. Further, the tubular member 102 may be configured in a variety of shapes, such as a continuous loop, multiple loops, or similar shapes.

FIG. 1B is a schematic view of the tubular member 102 shown in FIG. 1A, illustrating the opening 110, disposed proximate the proximal end 113 of the suction 111. The opening 110 may be arranged such that the actuation member 104 extending from the distal end 106 of the tubular member 102 may pass through the opening 110 and may further extend into and through the lumen 108 of the tubular member 102, finally extend outwards from the proximal end of the tubular member 102, to a position where it may be accessible to an operator and can be manipulated in order to deflect the tubular member 102. The opening 110 may extend to a specific depth within the tubular member 102, to facilitate its communicating with the lumen 108 of the tubular member 102. Moreover, the opening 110 may be circular in shape but other shapes including elliptical, ovular, rectangular, or the like may also be contemplated. The edges of opening 110 may be chamfered, beveled, rounded or include otherwise atraumatic configurations so as to reduce wear on actuation member 104 as it passes over the edges of opening 110.

FIG. 2 is a schematic illustration of the tubular member 102 of FIG. 1A, in a closed configuration, according to aspects of the present disclosure. When the actuation member 104 is pulled proximally or otherwise appropriately manipulated, the distal end 115 of the channel 111 may bent/moved towards the proximal end 113 of the suction channel 115 to form a loop 200 with channel 111 forming substantially surrounding an enclosed area. The distal loop 200 may be configured to engage a tissue layer within the circumference of the distal loop 200 via the channel 111. When the distal loop 200 is formed, the actuation member 104 may be retracted completely inside the lumen 108 of the tubular member 102 through opening 110.

In some embodiments, opening 110 may be formed through a slidable member or other appropriate longitudinally adjustable portion of tubular member 102, permitting adjustment of the loop 200 formed when the actuation member 104 is pulled proximally or otherwise appropriately manipulated. For example, a slidable member in accordance with one proposed embodiment could be actuated at a proximal end of the device by a pulling or pushing mechanism to either enlarge or decrease the diameter of the resulting loop 200. Such a mechanism may permit the loop 200 to properly encircle tissue targeted for resection.

In some embodiments, a distal portion of tubular member 102 may be formed of a shape memory material such as, e.g., Nitinol. In such embodiments, the distal portion of tubular member 102 may be configured to assume the closed, loop-like configuration when it is released from the confines of a constraining sheath (not shown), for example. It is contemplated, that the distal portion of tubular member 102 may be advanced to a desired site within a sheath (not shown), and the sheath may be retracted proximally (or the distal portion of tubular member 102 may be advanced distally out of the sheath), and the distal portion of tubular member 102 may assume its pre-formed configuration. In other embodiments, the distal portion of tubular member 102 may be configured to assume the closed, loop-like configuration upon exposure to certain triggers, including, but not limited to, body heat and/or body chemistry.

FIG. 3A shows a cutting assembly 300 including the longitudinal track 114 and the cutting tool 302 disposed thereon. As shown, the cutting tool 302 may be positioned inside the channel 111 of the tubular member 102, particularly, on or within the longitudinal track 114 defined in the channel 111. The track 114 may be positioned generally at the center of channel 111, and may further allow the cutting tool 302 to rest parallel along the longitudinal length of the tubular member 102 or the channel 111. The track 114 may limit cutting tool 302 to move only along the axis defined by the channel 111 and/or the tubular member 102. That is, the track 114 may also serve to limit radial travel of cutting tool 302 relative to track 114, so that a cutting depth cutting tool 302 into tissue may be controlled. The track 114 may provide a path for the cutting tool 302 to effectively grasp and/or resect tissue from within a patient's body. Further, the track 114 may include a surface, which may allow movement of the cutting tool 302 along the loop 200, facilitating circular incision in the tissue layer and/or resection of the tissue layer. To accomplish the cutting operation, any suitable tool such as a blade, scissor, needle knife, cautery wire, laser device or a combination of these may be used. It will be understood that other structures for maintaining the cutting tool 302 are conceivable and within the scope of the present disclosure.

In certain embodiments, the track 114 may include known mechanism that allows movement of the cutting tool 302 along the track 114. For example, the track 114 may include ring-like configurations, grooves, or similar geometrical structures disposed along the length of the track 114 to maneuver the cutting tool 302 or other tools disposed therein. These geometrical structures may be disposed at predetermined gaps along the length of the track 114.

In the depicted examples, the cutting tool 302 may be a sharp-edged blade that is slidably disposed on the longitudinal track 114. Specifically, the cutting tool 302 may include an actuation mechanism that moves or slides the cutting tool 302 along the longitudinal track 114. To facilitate such movement, the cutting tool 302 may be connected or integrated to a moveable element (not shown; such as control or actuation wire) so that the cutting tool 302 can traverse the length (or the circumference when the tubular member 102 is in the closed configuration) of the channel 111. In one example, the moveable element may be a flexible single control wire, for example, connected to the cutting tool 302. Specifically, a distal end of the moveable element may attach to the cutting tool 302, and a proximal end may be attached to the handle having an actuation mechanism such as a knob, lever, button, or other similar structure. When the operator activates the handle, such as by turning the knob, the moveable element may be pulled in a proximal direction, which may result in movement of the cutting tool 302 in the desired direction. In some embodiments, the handle may include a rotating mechanism adapted to rotate the cutting tool 302 such that tissue resection can be obtained.

In some embodiments, the moveable element may be contained within the longitudinal track 114 so that the moveable element is constricted radially but may travel along the longitudinal length of the tubular member 102 to actuate the cutting tool 302.

FIG. 3B shows the cutting tool 302 in operation. Through the actuation mechanism, the cutting tool 302 may be activated and further may be configured to slide at least partially along the length of the channel 111, to facilitate cutting the tissue layer lifted via the channel 111. Specifically, the cutting tool 302 may move about the circumference of the track 114, and may also be rotated upwards such that the sharp tip of the blade may be brought into contact with the circumference of the lifted tissue so as to facilitate effective incision and/or resection thereof. Once positioned appropriately, the sharp edge of a blade of cutting tool 302 may be used to resect the tissue layer in one continuous motion or the sharp edge may be used to first create a cut in the tissue layer, and then the cutting tool 302 may be moved incrementally in a step-wise fashion until the desired tissue layer is completely cut away. Herein, appropriate mechanisms may be used to facilitate upward rotation of the cutting tool 302, shown by the arrow 304, and its longitudinal movement along the arrow 306. In embodiments where the cutting tool 302 is an electrocautery device, suitable conductive mechanisms may be provided for conducting energy from a suitable source (e.g., an electrosurgical generator) to the cutting tool 302.

With tissue drawn into the channel 111, as shown in FIG. 4A, the operator may extend or move the cutting tool 302 along the longitudinal track 114 to create a circular incision in the tissue to resect the encircled tissue. Specifically, the cutting tool 302 may perform the cutting operation only for the tissue layer drawn into the suction channel 111, thereby enabling an operator to limit the cutting depth of cutting tool 302 to avoid inadvertently perforating or otherwise cutting or damaging underlying tissue layers.

When the cutting tool 302 is used to only make an incision, a separate cutting tool or snare or other device can be used in conjunction with the endoscope 10 to conduct resection and removal of the target tissue.

In exemplary embodiments, the cutting tool 302 may also include abrasive coatings or projections, such as barbs, saw teeth, or blades, although such protrusions should be sized to allow movement of the cutting tool 302 along the longitudinal track 114 with slot 109. Additionally, the cutting tool 302 employed in the present disclosure may be flexible in nature and adapted to be moved across and within the longitudinal track 114.

In the embodiment shown in FIG. 3C, the cutting tool 308 may be an electro-cautery wire, which may be conductive and may further be activated by passing electrical current through it to cut the tissue to be resected. It is envisioned that the cutting tool 308 may need to be electrically isolated from the rest of the device using the longitudinal track 114. The cautery wire can be configured as an actuation element and an electrical path for cautery cutting. For example, activation of the cutting tool 308 may be achieved by passing an electrical current through the cutting tool 308 such that the tissue in contact with the conductive portion is cut. For this embodiment, the cutting tool 308 may be connected to an electrocautery system (not shown) through suitable connections. In some embodiments, where the cutting tool 308 is an electro-cautery wire, the moveable element may be same as the actuation member 104, while in others, the moveable element may be a separate element.

The cutting tool 302 or 308 may be permanently fixed inside the channel 111, while in other embodiments, the cutting tool 302, 308 may be temporarily/removably disposed inside the suction channel 111.

It should be apparent that the resection device 100 of the present disclosure may be useful to perform surgical, diagnostic (such as, e.g., obtaining biopsy samples), and therapeutic procedures in a wide variety of bodily locations. For example, removal of polyps detected during a routine colonoscopy could quickly be accomplished using the devices and methods described herein. Additionally, stones or unwanted deposits can be engaged and removed from a variety of body lumens such as ureters, bladders, or the urethra. These and other procedures can be accomplished within the scope of the present disclosure.

FIG. 4A shows that resection device 100 may be placed against the target tissue that may be a part of or adjacent the tissue layer that is to be resected and/or retrieved. More specifically, FIG. 4A depicts the resection device 100, where the target tissue layer 402 can be encircled by tubular member 102 and channel 111 upon actuating the actuation member 104 at the proximal end of the tubular member 102. Then, suction may applied until the entire circumference of the channel 111 is in contact with the tissue layer 402. That is, tissue 402 is drawn into channel 111. Suctioning the tissue may help raise a circular ridge of the mucosal layer into slot 109, thereby separating it from the underlying tissue layers, including, but not limited to, the muscularis layer. With suction, the amount of tissue 402 drawn up into the channel 111 (i.e., into the depth of the channel) may be controlled by configuring the dimensions of channel 111 to only accept an amount of tissue that is limited to the mucosal layer, which may serve to reduce the risk of perforating underlying tissue layers. Once the appropriate amount of tissue is drawn into channel 111, the cutting tool 302 residing in the channel 111 may be activated and moved longitudinally along the channel 111 to create an incision 404 in the drawn-up tissue 402. The resulting incision 404 is shown in FIG. 4B. Creating the incision 404 may help the operator have a better grip of the desired tissue 402 to be resected, thereby addressing slippage problems as known in the art. Thereafter, a suitable cutting tool or the cutting tool 302 used for creating the incision 404 may facilitate the resection of the tissue layer 402.

Finally, as shown in FIG. 4C, a retrieval device 408 such as, e.g., a snare, graspers, tweezers, or forceps, may be extend through a lumen or working channel (e.g., lumen 108) of the tubular member 102, to retrieve the resected tissue portion from the patient's body. In some embodiments, a device such as snare may be embedded within the lumen 108 of the tubular member 102. The snare device may extend from the proximal end to the distal end 106, forming a loop inside the channel 111. When the snare device is drawn tight, the loop may be configured to extend out of the channel 111 into the circular incision so that it may ensare the tissue layer 402.

Exemplary Method

An exemplary method of using the resection device 100 is disclosed, according to one embodiment of the present disclosure. The method may be used in an exemplary Endoscopic Mucosal Resection (EMR) procedure at a target location within a patient's body. The target location may be a body cavity including two or more layers of tissue such as a mucosal layer and a submucosal layer (e.g., a muscularis layer). In the current embodiment, it can be considered that a part of a mucosal layer may contain cancerous polyps, lesions or other undesired tissues to be resected.

Initially, an operator may insert the elongate sheath 12 within a patient's body through natural anatomical openings such as the mouth, nose, anus, ureter, vaginal cavity, or the like. As noted above, the elongate sheath 12 may also be inserted into a patient's body through an incision. After inserting the sheath 12, the operator may maneuver the sheath 12 through the patient's body to reach the target location. An illumination device along with an optical/visualization device may facilitate maneuvering of the sheath 12 during the resection procedure. The illumination, optical, and/or visualization devices may be integral with sheath 12, or may be auxiliary devices disposed in one or more working channels of sheath 12. Next, the operator may distally extend the resection device 100 from the sheath 12 and position it over the tissue portion to be resected.

While the resection device 100 is inserted, the tubular member 102 may remain in a straightened configuration having one end of the actuation member 104 attached to the distal end 106, and other end connected to a handle (not shown) or other suitable actuator (also not shown). Here, the tubular member 102 may also include the cutting tool 302 disposed in the channel 111.

Thereafter, the tubular member 102 may be moved to a closed configuration using the actuation member 104 coupled to the tubular member 102. When proximal forces are applied by the actuation member 104 to the distal end 106 of tubular member 102, the distal end 106 of the tubular member 102 may move towards the proximal end 113 of the channel 111, thereby forming a loop with a circular channel 111, to encircle a desired tissue portion. The actuation may also move the cutting tool 302 towards the created loop. Further, the tubular member 102 can be adjusted such that a size of the created loop may be adjusted so that the loop may cover/surround a substantial entirety of the targeted tissue. Such adjustment may be achieved by the application of proximal or distal forces one or more times on the tubular member 102 using the actuation member 104.

Once the tubular member 102 surrounds/covers the targeted tissue, suction force may be applied through the lumen 108 to create a tissue ridge by drawing the tissue 402 into channel 111. Through the suction, a tissue layer that is to be removed (or immediately adjacent tissue) may be lifted with the help of the channel 111, thereby separating the top most layer (e.g., mucosa) from an underlying layer (e.g., muscularis). During this process, the depth of the tissue layer pulled into the channel 111 may determine the depth of the cut to be made by the cutting tool 302. Then, the tissue layer drawn into the channel 111 may be manipulated using the cutting tool 302 disposed in the channel 111. Specifically, the cutting tool 302 may be activated and moved in the desired direction, to enable cutting of the tissue 402 that has been drawn into the suction channel 111.

In some embodiments, the cutting tool 302 may be initially used to selectively cut the tissue to make perimeter cuts on the tissue layer to be resected. Once the perimeter incision is created, the same cutting tool 302 may be used to resect the tissue layer through the incision. In some cases, however, an additional cutting tool may be inserted through the lumen 108 of the tubular member 102 to resect and/or remove the tissue layer.

Subsequently, an additional device such as a snare, forceps, grasper, or a basket, may be inserted through the lumen 108 or other working channel of the tubular member 102 to retrieve the resected tissue layer 402 from the body lumen. Finally, when the medical procedure is completed, the operator may then retract the resection device 100 within the sheath 12 and then may retract the sheath 12 from the patient's body.

In embodiments where the cutting tool 302 is an electrocautery device, no further steps may be necessary to close any open tissue interfaces. However, in other embodiments, the incision created by cutting tool 302 may be appropriately closed by, for example, sutures or staples, as is known in the art.

It may be apparent to a person skilled in the art that the teachings of the present disclosure as illustrated above are not limited to the above embodiments. In fact, the teachings of the present disclosure can be carried out using alternative embodiments.

Advantages and characteristics of the embodiments include devices and methods for resecting surface tissue layers in an easy and safe manner, while preventing damage to the surrounding and/or underlying tissue layers. Further, the disclosed devices have the ability to expand to a certain degree and capture only the desired tissue layer to be resected. Embodiments of the present disclosure may be used in various different medical or non-medical procedure, including medical procedures where appropriate resection of undesired body tissue is required. In addition, at least certain aspects of the aforementioned embodiments may be combined with other aspects of the embodiments, or removed, without departing from the scope of the disclosure.

Other embodiments of the present disclosure will be apparent to those skilled in the art from consideration of the specification and practice of the embodiments disclosed herein. It is intended that the specification and examples be considered as exemplary only, with a true scope and spirit of the invention being indicated by the following claims.

What is claimed is:

1. A medical device, comprising:
a tubular member having a proximal end, a distal end, and a lumen extending therebetween, the tubular member including an opening at a distal portion of the tubular member, wherein the opening is in communication with the lumen, wherein a distal end of the opening is disposed proximate of the distal end of the tubular member, and wherein the distal portion of the tubular member is configured to bend in at least one plane; and
an actuation member extending at least partially within the tubular member, wherein a distal portion of the actuation member is configured to exit the tubular member at a location proximate the opening, wherein a distal end of the actuation member is coupled to the distal end of the tubular member.

2. The medical device of claim 1, further comprising a cutting tool disposed within the opening.

3. The medical device of claim 1, wherein the opening includes an elongate configuration.

4. The medical device of claim 2, wherein the cutting tool is an electro-cautery tool.

5. The medical device of claim 2, wherein the cutting tool is configured to traverse the opening.

6. The medical device of claim 1, wherein the lumen is configured to be coupled to a suction source.

7. The medical device of claim 1, wherein the opening extends distally from the location where the actuation member exits the tubular member.

8. The medical device of claim 1, wherein an external surface of the distal portion includes a plurality of radiopaque markings.

9. The medical device of claim 2, wherein the cutting tool is slidably disposed on a track that traverses the opening.

10. The medical device of claim 1, wherein pulling the actuation member proximally is configured to cause the distal portion of tubular member to form a loop with itself.

11. A medical device, comprising:
an elongate tubular member having a proximal end, a distal end, and a lumen extending therebetween, wherein a distal end portion of the elongate tubular member is configured to bend towards a remaining portion of the elongate member to create a loop-like configuration, and wherein the distal end portion includes an elongate rectangular opening in communication with the lumen; and
an actuation member coupled to a distal end face of the tubular member, wherein pulling the actuation member proximally causes the distal end portion of the elongate tubular member to bend towards the remaining portion.

12. The medical device of claim 11, further comprising a cutting tool disposed within the opening.

13. The medical device of claim 12, wherein the cutting tool is an electro-cautery tool.

14. The medical device of claim 12, wherein the cutting tool is configured to traverse the opening.

15. The medical device of 11, wherein the lumen is configured to be coupled to a suction source.

16. The medical device of claim 11, wherein a portion of the actuation member is disposed within the elongate tubular member, and a portion of the actuation member is disposed outside of the elongate tubular member.

17. The medical device of claim 11, wherein an external surface of the distal end portion includes a plurality of radiopaque markings.

18. The medical device of claim 12, wherein the cutting tool is slidably disposed on a track that traverses the opening.

19. A method of resecting tissue, comprising:
positioning a medical device adjacent tissue targeted for resecting, wherein the medical device includes:
an elongate tubular member having a proximal end, a distal end, and a lumen extending therebetween, wherein a distal end portion of the elongate tubular member is configured to bend towards a remaining portion of the elongate tubular member to create a loop-like configuration, and wherein the distal end portion includes an elongate rectangular opening in communication with the lumen; and
an actuation member coupled to a distal end face of the elongate tubular member, wherein pulling the actuation member proximally causes the distal end portion of the elongate tubular member to bend towards the remaining portion of the elongate tubular member;
pulling the actuation member to bend the distal end portion of the elongate tubular member towards the remaining portion of the elongate tubular member to create a substantially loop-like configuration;
drawing tissue into the elongate rectangular opening, and activating a cutting tool to cut through the tissue drawn into the elongate rectangular opening, wherein the tissue drawn into the elongate rectangular opening may include only a mucosal layer of tissue.

20. The method of claim 19, further comprising retrieving the resected tissue.
